(19) United States
(12) Patent Application Publication
(10) Pub. No.: US 2014/0230522 A1
(43) Pub. Date: Aug. 21, 2014

(54) CLEANLINESS MEASURING CARRIAGE AND CLEANLINESS MEASURING SYSTEM

(71) Applicant: Kabushiki Kaisha Tokshiba, Tokyo (JP)

(72) Inventors: Eri UEMURA, Chiba (JP); Makiko KATANO, Kanagawa (JP); Tsunekazu YASUTAKE, Kanagawa (JP)

(73) Assignee: KABUSHIKI KAISHA TOSHIBA, Tokyo (JP)

(21) Appl. No.: 14/013,389

(22) Filed: Aug. 29, 2013

(30) Foreign Application Priority Data
Feb. 18, 2013 (JP) 2013-029088

(51) Int. Cl. G01N 33/00

(52) U.S. Cl. CPC G01N 33/0009
USPC 73/28.01

(57) ABSTRACT

A cleanliness measuring carriage includes a cleanliness measuring device for measuring cleanliness within a semiconductor manufacturing room, a holding unit for holding the cleanliness measuring device, and a conveying unit for conveying the cleanliness measuring device along a substrate container transport lane.

### CROSS-REFERENCE TO RELATED APPLICATION

This application is based upon and claims the benefit of priority from Japanese Patent Application No. 2013-029088, filed Feb. 18, 2013, the entire contents of which are incorporated herein by reference.

### FIELD

Embodiments described herein relate generally to a cleanliness measuring carriage and a cleanliness measuring system.

### BACKGROUND

A semiconductor manufacturing clean room currently used includes a plurality of fixed-point monitors that are disposed at uniform intervals and in communication with a particle counter to measure particles (contaminants) within the clean room. It is difficult, however, to specify the exact location of a particle generating source by using the fixed-point monitors. A movable particle counter is known, but this type of counter requires human labor to be conveyed to a measurement position and therefore is difficult to measure particles at appropriate times. Accordingly, prompt determination of the location of the particle generating source is difficult when the fixed-point monitor in the related art is used.

### DESCRIPTION OF THE DRAWINGS

FIG. 1 schematically illustrates the general structure of a cleanliness measuring system according to a first embodiment.

FIG. 2 schematically illustrates the general structure of a cleanliness measuring system according to a second embodiment.

FIG. 3 schematically illustrates a cleanliness measuring method according to a third embodiment.

FIG. 4 schematically illustrates an application example of the cleanliness measuring method according to the third embodiment.

### DETAILED DESCRIPTION

According to an embodiment, there is provided a cleanliness measuring carriage and a cleanliness measuring system capable of determining a location of a particle generating source.

In general, according to one embodiment, a cleanliness measuring carriage includes a cleanliness measuring device for measuring a cleanliness level within a semiconductor manufacturing room, a holding unit holding the cleanliness measuring device, and a conveying unit conveying the cleanliness measuring device along a conveying lane otherwise provided for transfer of a wafer conveying container.

Embodiments are hereinafter described with reference to the drawings.

### First Embodiment

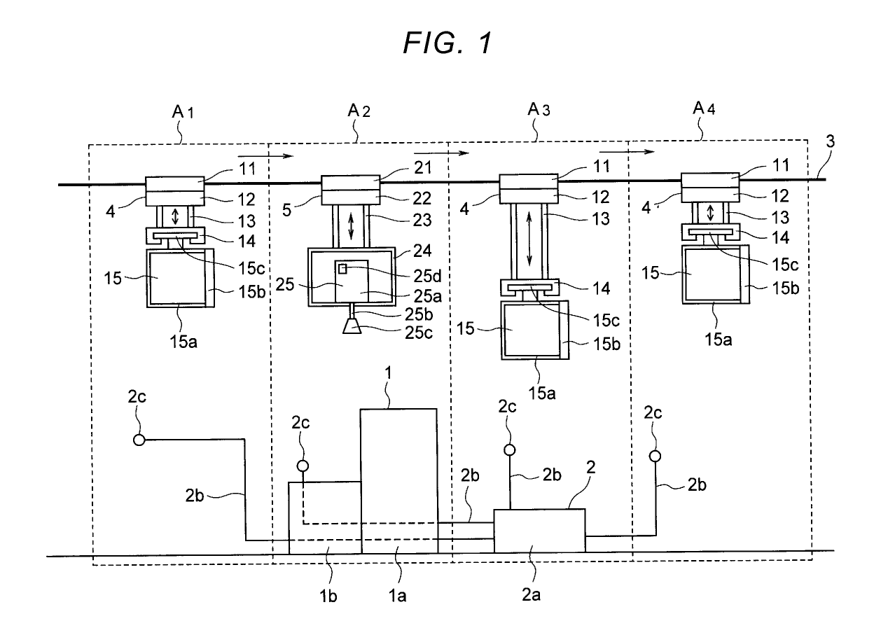

FIG. 1 schematically illustrates the general structure of a cleanliness measuring system according to a first embodiment. The cleanliness measuring system shown in FIG. 1 is positioned within a semiconductor manufacturing clean room.

FIG. 1 shows the semiconductor manufacturing clean room as viewed from the side.

The cleanliness measuring system illustrated in FIG. 1 includes a semiconductor manufacturing device 1, a fixed-point monitor 2, a FOUP (front opening unified pod) conveying lane 3, FOUP conveying carriages 4, and a cleanliness measuring carriage 5.

The semiconductor manufacturing device 1 is a device which manufactures a semiconductor device on a semiconductor wafer. The semiconductor manufacturing device 1 is constituted by an EFEM (equipment front end module), for example. The semiconductor manufacturing device 1 has a main unit 1a and a FOUP load port 1b.

The fixed-point monitor 2 is a device which measures cleanliness at plural measuring positions within the semiconductor manufacturing clean room. The fixed-point monitor 2 is an example of a cleanliness monitor in this disclosure. The fixed-point monitor 2 has a particle counter 2a, a plurality of tubes 2b connected with the particle counter 2a, and a plurality of sensor devices 2c attached to the tips of the respective tubes 2b.

The sensor devices 2c are provided at uniform length intervals within the semiconductor manufacturing clean room. FIG. 1 illustrates a condition in which the one sensor device 2c is equipped in each of measurement areas A₁ through A₄ within the semiconductor manufacturing clean room.

The particle counter 2a measures the amount of particles (such as number and concentration of particles) at the installation positions of the sensor devices 2c to determine the cleanliness therein. The particle counter 2a measures the number and concentration of the particles in the vicinity of the sensor device 2c within the measurement area A₁ by using the sensor device 2c equipped in the measurement area A₁, for example.

The FOUP conveying lane 3 is a conveying (transfer) lane provided for movement of FOUPs within the semiconductor manufacturing clean room. Each of the FOUPs is a conveying container (substrate container) used for conveying a semiconductor wafer, corresponding to an example of a wafer conveying container in this disclosure. The FOUP conveying lane 3 in this embodiment is used not only for the travel of the FOUP conveying carriages 4, but also for the travel of the cleanliness measuring carriage 5. The FOUP conveying lane 3 in this embodiment is of a ceiling travel type, i.e., the FOUP carriers are supported from above and moved between process locations along the ceiling of the facility, but it may be of other types (such as floor travel type).

Each of the FOUP conveying carriages 4 is a carriage which automatically conveys a FOUP 15. The FOUP conveying carriage 4 includes a conveying unit 11, an elevating mechanism 12, an elevating belt 13, and an FOUP holding unit 14. The FOUP 15 has a FOUP main body 15a, a door 15b, and a flange 15c.

The conveying unit 11 engages with the FOUP conveying lane 3, and conveys the FOUP 15 along the FOUP conveying lane 3. The elevating mechanism 12 shifts the FOUP 15 upward and downward relative to the FOUP conveying lane 3 by shortening and lengthening of the elevating belt 13. The FOUP holding unit 14 holds the FOUP 15 by gripping the flange 15c. The door 15b of the FOUP 15 is opened and closed when the semiconductor wafer is put into or taken out of the FOUP main body 15a.

The cleanliness measuring carriage 5 is a carriage which automatically conveys a cleanliness measuring device 25. The cleanliness measuring carriage 5 includes a conveying unit 21, an elevating mechanism 22, an elevating belt 23, and a holding unit 24. The cleanliness measuring device 25 includes a device main body 25a, a tube 25b connected with the device main body 25a, a suction port 25c attached to the tip of the tube 25b, and a communication unit 25d provided on the device main body 25a.

The conveying unit 21 engages with the FOUP conveying lane 3, and conveys the cleanliness measuring device 25 along the FOUP conveying lane 3. The elevating mechanism 22 shifts the cleanliness measuring device 25 upward and downward relative to the FOUP conveying lane 3 by shortening and lengthening of the elevating belt 23. The elevating mechanism 22 and the elevating belt 23 are but one example of an elevating unit according to this disclosure. The holding unit 24 holds the cleanliness measuring device 25 by supporting the cleanliness measuring device 25 within the holding unit 24.

The holding unit 24 may have a door through which the cleanliness measuring device 25 is put into and taken out of the holding unit 24. The holding unit 24 may hold the cleanliness measuring device 25 by gripping a flange of the cleanliness measuring device 25 instead of supporting the cleanliness measuring device 25, for example. Furthermore, the holding unit 24 may secure the cleanliness measuring device 25 in such a manner that the cleanliness measuring device 25 is detachably attached to the holding unit 24, or may secure the cleanliness measuring device 25 in such a manner that the cleanliness measuring device 25 is fixed to the holding unit 24.

The cleanliness measuring device 25 is a device which measures cleanliness within the semiconductor manufacturing clean room. The cleanliness measuring carriage 5 in this embodiment measures the cleanliness in the space through which it is moved using the cleanliness measuring device 25 while moving the cleanliness measuring device 25 into different regions of the semiconductor manufacturing clean room. According to this structure, the cleanliness measuring carriage 5 in this embodiment can automatically specify the location of the contaminant source within the semiconductor manufacturing clean room.

The power for the cleanliness measuring device 25 is supplied from a battery, for example. The battery is charged while the cleanliness measuring device 25 is not conducting measurements.

The device main body 25a of the cleanliness measuring device 25 is constituted by a particle counter, for example. In this case, the device main body 25a measures the amount of particles (number or concentration of particles) sucked through the suction port 25c to determine the amount of particles. The particle counter is constituted by a laser optical counter or a condensation particle counter, for example.

The tube 25b passes through a hole formed in the holding unit 24, allowing the suction port 25c to be positioned outside the holding unit 24. The cleanliness measuring carriage 5 in this embodiment varies the height of the suction port 25c within the processing space or clean room by lengthening and shortening the elevating belt 23 so as to control the particle measurement position in the height direction within the different regions of the semiconductor manufacturing clean room.

The communication unit 25d is provided for communication between the cleanliness measuring device 25 and other devices. The communication unit 25d is located inside the device main body 25a in this embodiment, but may be disposed outside the device main body 25a.

### (Operation of Cleanliness Measuring System in First Embodiment)

The operation of the cleanliness measuring system according to the first embodiment is now explained in detail with reference to FIG. 1.

After measuring the cleanliness within the semiconductor manufacturing clean room, the fixed-point monitor 2 transmits to the cleanliness measuring carriage 5 measurement data of the cleanliness measured by the fixed-point monitor 2, and position information for specifying the measurement location, i.e., the position of the carriage 5 where this cleanliness is measured.

The measurement data contains the number or concentration of the measured particles, for example. The measurement data may include particle concentrations, or indexes at plural levels (such as indexes in three levels consisting of “low level”, “medium level”, and “high level”) showing particle concentrations, for example.

The position information contains information about the location of the sensor device 2c used for the cleanliness measurement, and information about the position of any one of the measurement areas A₁ through A₄ where the cleanliness is measured, for example. The position information may be other information as long as the information can specify the measurement location where the cleanliness is measured.

The fixed-point monitor 2 may transmit the measurement data and the position information to either the communication unit 25d of the cleanliness measuring device 25, or a communication unit provided on a control unit (not shown) of the cleanliness measuring carriage 5. The control unit of the cleanliness measuring carriage 5 is equipped within the conveying unit 21, for example, and controls the lateral movement of the conveying unit 21, the upward and downward movement of the elevating mechanism 22 and the operation of the elevating belt 23, and other operations.

Upon receiving the measurement data and the position information from the fixed-point monitor 2, the cleanliness measuring carriage 5 determines the position of the cleanliness measuring device 25 based on these measurement data and position information. The cleanliness measuring carriage 5 then moves to the region where the number of particles measured by the fixed-point monitor 2 is high in accordance with the measurement data and the position information. The cleanliness measuring carriage 5 having reached this region initiates measurement of the particle concentration by using the cleanliness measuring device 25 while moving around the region.

Accordingly, the cleanliness measuring system in this embodiment can specify the rough location of the particle generating source by using the fixed-point monitor 2, and then determine the detailed location of the particle generating source by using the cleanliness measuring carriage 5.

Moreover, the cleanliness measuring carriage 5 in this embodiment controls the conveying speed of the cleanliness measuring device 25 based on the cleanliness data measured by the cleanliness measuring device 25. For example, the cleanliness measuring carriage 5 increases the conveying speed when the particle concentration under measurement is low, and decreases the conveying speed when the particle concentration under measurement is high.

This speed control of the cleanliness measuring carriage 5 in this embodiment allows more detailed monitoring by the cleanliness measuring carriage 5 for the region where the particle concentration is high, thereby allowing rapid determination of the detailed location of the particle generating source.

The cleanliness measuring carriage 5 may set to be conveyed at a conveying speed at either one of two levels of a “low speed” and a “high speed”, or the conveying speed may be set at any one of three or more speed levels.

The cleanliness measuring carriage 5 should maintain a certain distance between the FOUP conveying carriages 4 and the cleanliness measuring carriage 5 during movement so as to avoid contact between the FOUP conveying carriages 4 and the cleanliness measuring carriage 5.

After measuring the cleanliness level within the semiconductor manufacturing clean room, the cleanliness measuring device 25 transmits the measurement data of the cleanliness level measured by the cleanliness measuring device 25, and the position information for specifying the measurement location where the cleanliness level is measured, via the communication unit 25d, as necessary.

The measurement data contains the number or concentration of the measured particles, for example. The measurement data may include particle concentrations, or indexes at plural levels showing particle concentrations.

The position information contains information about the location of the cleanliness measuring carriage 5 or the height of the cleanliness measuring device 25 at the time of measurement of the cleanliness, for example. The position information may be other information as long as the information can specify the measurement position corresponding to where the cleanliness is measured.

After specifying the region where the particle concentration is high, for example, the cleanliness measuring device 25 in this embodiment transmits the position information and the measurement data about the corresponding area via the communication unit 25d. These position information and measurement data are sent to a PC (personal computer) or other device of a manager of the semiconductor manufacturing clean room or the cleanliness measuring system, for example. Based on the information and data, the manager can take measures such as stopping the semiconductor manufacturing processes and removing the contaminant source.

As discussed above, the cleanliness measuring system in this embodiment measures the cleanliness within the semiconductor manufacturing clean room by using the cleanliness measuring device 25 as it is moved by the cleanliness measuring carriage 5 and facility conveying system.

Accordingly, unlike the measurement of cleanliness using only a fixed cleanliness measuring device and the measurement of cleanliness using a manually movable cleanliness measuring device, the cleanliness measuring system in this embodiment can automatically relocate the cleanliness measuring device 25 to a desired measurement position.

According to this embodiment, therefore, the position of the contaminant source within the semiconductor manufacturing clean room can be promptly determined. This advantage can reduce defects produced by adhesion of particles to the semiconductor wafer.

### Second Embodiment

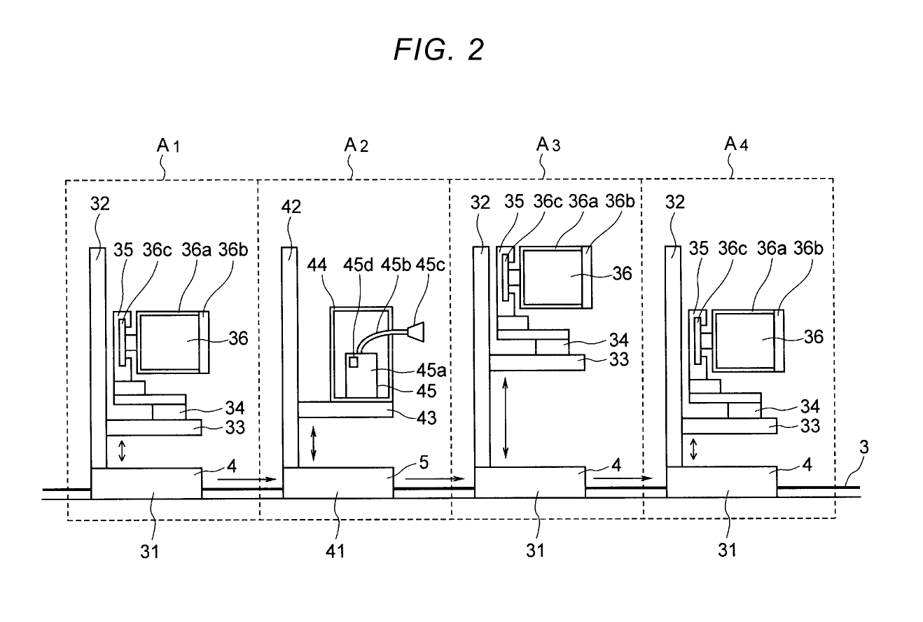

FIG. 2 schematically illustrates the general structure of a cleanliness measuring system according to a second embodiment.

The FOUP conveying lane 3 shown in FIG. 1 is of a ceiling traveling type. On the other hand, the FOUP conveying lane 3 shown in FIG. 2 is of a floor traveling type. FIG. 2 does not show the semiconductor manufacturing device 1 and the fixed-point monitor 2 of FIG. 1 for easier understanding of the figure.

Each of the FOUP conveying carriages 4 shown in FIG. 2 is a carriage which automatically conveys a FOUP 36, and includes a conveying unit 31, a crane unit 32, a base unit 33, an arm unit 34, and a hand unit 35. The FOUP 36 includes a FOUP main body 36a, a door 36b, and a flange 36c.

The conveying unit 31 engages with the FOUP conveying lane 3, and conveys the FOUP 36 along the FOUP conveying lane 3. The crane unit 32 moves the FOUP 36 upward and downward relative to the FOUP conveying lane 3 by the upward and downward movement of the base unit 33. The hand unit 35 is attached to the base unit 33 via the arm unit 34, and holds the FOUP 36 by gripping the flange 36c. The door 36b of the FOUP 36 is opened and closed when the semiconductor wafer is put into or taken out of the FOUP main body 36a.

The cleanliness measuring carriage 5 shown in FIG. 2 is a carriage which automatically conveys a cleanliness measuring device 45, and includes a conveying unit 41, a crane unit 42, a base unit 43, and a holding unit 44. The cleanliness measuring device 45 has a device main body 45a, a tube 45b connected with the device main body 45a, a suction port 45c attached to the tip of the tube 45b, and a communication unit 45d provided on the device main body 45a.

The conveying unit 41 engages with the FOUP conveying lane 3, and conveys the cleanliness measuring device 45 along the FOUP conveying lane 3. The crane unit 42 moves the cleanliness measuring device 45 upward and downward relative to the FOUP conveying lane 3 by the upward and downward movement of the base unit 43. The crane unit 42 and the base unit 43 are an example of the elevating unit in this disclosure. The holding unit 44 holds the cleanliness measuring device 45 by accommodating the cleanliness measuring device 45.

The cleanliness measuring device 45 is a device which measures cleanliness within the semiconductor manufacturing clean room. The cleanliness measuring carriage 5 in this embodiment measures the cleanliness by using the cleanliness measuring device 45 while moving the cleanliness measuring device 45. This structure allows the cleanliness measuring carriage 5 in this embodiment to automatically identify the location of the contaminant source within the semiconductor manufacturing clean room.

The details of the elements in FIG. 2 that are similar to the corresponding elements in the first embodiment described in FIG. 1 are not repeatedly explained herein for brevity.

According to this embodiment, the position of the contaminant source within the semiconductor manufacturing clean room can be rapidly determined similarly to the first embodiment.

### Third Embodiment

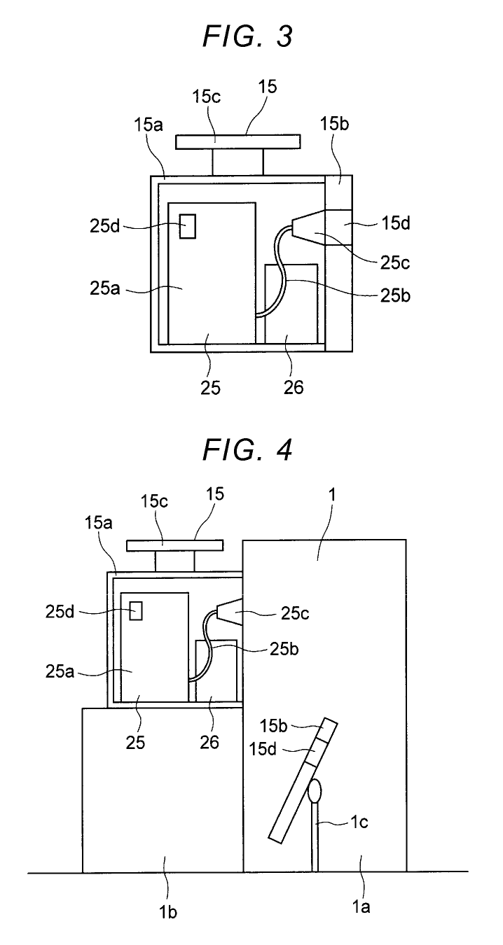

FIG. 3 schematically illustrates a cleanliness measuring method according to a third embodiment.

The cleanliness measuring system in the first embodiment conveys the cleanliness measuring device 25 by using the cleanliness measuring carriage 5 provided separately from the FOUP conveying carriages 4. As an alternative, the cleanliness measuring system in the third embodiment conveys the cleanliness measuring device 25 by using the FOUP conveying carriage 4 (shown in FIG. 1). In other words, the FOUP conveying carriage 4 also functions as a cleanliness measuring carriage according to the cleanliness measuring system in the third embodiment.

As illustrated in FIG. 3, the cleanliness measuring device 25 in this embodiment is accommodated in the FOUP 15. The FOUP 15 containing the cleanliness measuring device 25 is held by the FOUP holding unit 14 (FIG. 1), and conveyed by the conveying unit 11 (FIG. 1). The conveying unit 11 and the FOUP holding unit 14 are examples of the conveying unit and the holding unit in this disclosure, respectively. In addition, the elevating mechanism 12 and the elevating belt 13 in this structure are an example of the elevating unit in this disclosure.

The door 15b of the FOUP 15 shown in FIG. 3 has a opening 15d. The tube 25b of the cleanliness measuring device 25 shown in FIG. 3 is fixed in a position whereby the suction port 25c faces the opening 15d. According to this structure, the cleanliness measuring device 25 can measure the cleanliness within the semiconductor manufacturing clean room with the door 15b closed.

The FOUP 15 shown in FIG. 3 also contains a measuring device 26 as a measuring component different from the cleanliness measuring device 25. The measuring device 26 is a differential pressure gauge, an anemometer, or an ammonia monitor, for example.

The cleanliness measuring method in this embodiment may also be utilized with the FOUP 36 in the second embodiment. In this case, the conveying unit 31 is an example of the conveying unit in this disclosure. The crane unit 32 and the base unit 33 are an example of the elevating unit in this disclosure. The arm unit 34 and the hand unit 35 are an example of the holding unit in this disclosure.

FIG. 4 schematically illustrates an application example of the cleanliness measuring method according to the third embodiment.

According to the cleanliness measuring system in this embodiment, the FOUP 15 containing the cleanliness measuring device 25 moves downward toward the FOUP load port 1b of the semiconductor manufacturing device 1 to be loaded on the FOUP load port 1b as illustrated in FIG. 4. In this condition, the door 15b of the FOUP 15 is automatically removed by a FOUP opener 1c of the semiconductor manufacturing device 1.

According to the cleanliness measuring system in this embodiment, the FOUP 15 containing the cleanliness measuring device 25 lies on the FOUP load port 1b, allowing the cleanliness measuring device 25 to measure the cleanliness inside the semiconductor manufacturing device 1.

Moreover, according to the cleanliness measuring system in this embodiment, measurement of the cleanliness inside all the semiconductor manufacturing devices 1 provided within the semiconductor manufacturing clean room can be achieved by utilizing the function of the automatic conveyance of the FOUP 15 along the FOUP conveying lane 3.

The position of the suction port 25c of the cleanliness measuring device 25 is fixed to the door 15b side of the FOUP so that the cleanliness within the semiconductor manufacturing device 1 can be easily measured.

When the measuring device 26 is provided as a differential pressure gauge, the measuring device 26 can monitor pressure between the inside and outside of the semiconductor manufacturing device 1. When a differential pressure exists between the inside and outside of the semiconductor manufacturing device 1, particles may enter the interior of the semiconductor manufacturing device 1 and contaminate the semiconductor manufacturing device 1. Accordingly, when the measuring device 26 detects differential pressure, the cleanliness measuring system in this embodiment may stop the use of the corresponding semiconductor manufacturing device 1, or transmit a notification to the PC, or the like, of the manager about the detection of the differential pressure.

When the measuring device 26 is provided as an anemometer, the measuring device 26 can measure airflow within the semiconductor manufacturing device 1. When airflow exists in the semiconductor manufacturing device 1, particles may enter the interior of the semiconductor manufacturing device 1 and contaminate the semiconductor manufacturing device 1. Accordingly, when the measuring device 26 detects airflow, the cleanliness measuring system in this embodiment may stop the use of the corresponding semiconductor manufacturing device 1, or transmit a notification to the PC, or the like, of the manager about the detection of the airflow.

When the measuring device 26 is provided as an ammonia monitor, the measuring device 26 can measure ammonia levels within the semiconductor manufacturing device 1 or within the semiconductor manufacturing clean room. Ammonia contained in the semiconductor manufacturing device 1 is generally removed by a filter. However, when this filter is broken or used longer than the service life thereof, the device 1 or the clean room may be filled with ammonia. Accordingly, when the measuring device 26 detects ammonia within the device 1 or within the clean room, the cleanliness measuring system in this embodiment may stop the semiconductor manufacturing process, or transmit a notification to the PC, or the like, of the manager about the detection of the ammonia.

As discussed above, the cleanliness measuring system in this embodiment conveys the cleanliness measuring device 25 using the FOUP 15. Accordingly, any abnormal conditions within the semiconductor manufacturing device 1 can be promptly detected based on the measurement of the cleanliness within the semiconductor manufacturing device 1 according to this embodiment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cleanliness measuring carriage, comprising:
a cleanliness measuring device for monitoring cleanliness within a semiconductor manufacturing room;
a holding unit for supporting the cleanliness measuring device; and
a conveying unit for transferring the cleanliness measuring device along a substrate container transport lane.

2. The cleanliness measuring carriage according to claim 1, wherein the conveying unit controls the conveying speed of the cleanliness measuring device based on the cleanliness level of the semiconductor manufacturing room.

3. The cleanliness measuring carriage according to claim 2, further comprising:
an elevating unit moving the cleanliness measuring device upward and downward relative to the substrate container transport lane.

4. The cleanliness measuring carriage according to claim 3, further comprising:
a communication unit transmitting contamination data measured by the cleanliness measuring device, and location information for specifying a measurement position where the data is obtained.

5. The cleanliness measuring carriage according to claim 3, wherein the semiconductor manufacturing room comprises a fixed point cleanliness monitor measuring cleanliness at a plurality of locations within the semiconductor manufacturing room, and
the conveying unit determines a position of the cleanliness measuring device based on the position information for specifying a location where the data is obtained.

6. The cleanliness measuring carriage according to claim 3, wherein the holding unit comprises a substrate conveying container having the cleanliness measuring device disposed therein, and the conveying unit is used to transfer the substrate conveying container along the substrate container transport lane.

7. The cleanliness measuring carriage according to claim 2, further comprising:
a communication unit transmitting contamination data measured by the cleanliness measuring device, and location information for specifying a measurement position where the data is obtained.

8. The cleanliness measuring carriage according to claim 7, wherein the semiconductor manufacturing room comprises a fixed point cleanliness monitor measuring cleanliness at a plurality of locations within the semiconductor manufacturing room, and
the conveying unit determines a position of the cleanliness measuring device based on contamination data measured by the fixed point cleanliness monitor and position information for specifying a location where the data is obtained.

9. The cleanliness measuring carriage according to claim 8, wherein the holding unit comprises a substrate conveying container having the cleanliness measuring device disposed therein, and the conveying unit is used to transfer the substrate conveying container along the substrate container transport lane.

10. The cleanliness measuring carriage according to claim 1, further comprising:
an elevating unit moving the cleanliness measuring device upward and downward relative to the substrate container transport lane.

11. The cleanliness measuring carriage according to claim 10, further comprising:
a communication unit transmitting contamination data measured by the cleanliness measuring device, and location information for specifying a measurement position where the data is obtained.

12. The cleanliness measuring carriage according to claim 10, wherein the semiconductor manufacturing room comprises a fixed point cleanliness monitor measuring cleanliness at a plurality of locations within the semiconductor manufacturing room, and
the conveying unit determines a position of the cleanliness measuring device based on contamination data measured by the fixed point cleanliness monitor and position information for specifying a location where the data is obtained.

13. The cleanliness measuring carriage according to claim 10, wherein the holding unit comprises a substrate conveying container having the cleanliness measuring device disposed therein, and the conveying unit is used to transfer the substrate conveying container along the substrate container transport lane.

14. The cleanliness measuring carriage according to claim 1, further comprising:
a communication unit transmitting contamination data measured by the cleanliness measuring device, and location information for specifying a measurement position where the data is obtained.

15. The cleanliness measuring carriage according to claim 14, wherein the semiconductor manufacturing room comprises a fixed point cleanliness monitor measuring cleanliness at a plurality of locations within the semiconductor manufacturing room, and
the conveying unit determines a position of the cleanliness measuring device based on contamination data measured by the fixed point cleanliness monitor and position information for specifying a location where the data is obtained.

16. The cleanliness measuring carriage according to claim 14, wherein the holding unit comprises a substrate conveying container having the cleanliness measuring device disposed therein, and the conveying unit is used to transfer the substrate conveying container along the substrate container transport lane.

17. The cleanliness measuring carriage according to claim 1, wherein the semiconductor manufacturing room comprises a fixed point cleanliness monitor measuring cleanliness at a plurality of locations within the semiconductor manufacturing room, and
the conveying unit determines a position of the cleanliness measuring device based on contamination data measured by the fixed point cleanliness monitor and position information for specifying a location where the data is obtained.

18. The cleanliness measuring carriage according to claim 1, wherein the holding unit comprises a substrate conveying container having the cleanliness measuring device disposed therein, and the conveying unit is used to transfer the substrate conveying container along the substrate container transport lane.

19. A cleanliness measuring system, comprising:
a conveying lane provided within a semiconductor manufacturing room;
a substrate conveying carriage adapted to travel along the conveying lane to convey a substrate conveying container; and
a cleanliness measuring carriage adapted to travel along the conveying lane,
wherein the cleanliness measuring carriage comprises:
a cleanliness measuring device for monitoring cleanliness within the semiconductor manufacturing room;
a holding unit for supporting the cleanliness measuring device;
a conveying unit for conveying the cleanliness measuring device along a substrate container transport lane.

20. The cleanliness measuring system according to claim 19, wherein the cleanliness measuring carriage comprises a substrate conveying container having the cleanliness measuring device disposed therein, and the conveying unit is used to transfer the substrate conveying container along the substrate container transport lane.
